(19) United States
(12) Patent Application Publication
(10) Pub. No.: US 2023/0145281 A1
(43) Pub. Date: May 11, 2023

(54) SYSTEM AND METHOD FOR PREDICTING RISK OF DIAGNOSIS FOR AUTISM SPECTRUM DISORDER USING NEONATAL ANALYTICS

(71) Applicant: University of Virginia Patent Foundation, Charlottesville, VA (US)

(72) Inventors: KAREN D. FAIRCHILD, Charlottesville, VA (US); Douglas E. LAKE, Charlottesville, VA (US)

(21) Appl. No.: 17/909,170

(22) PCT Filed: Mar. 8, 2021
(86) PCT No.: PCT/US21/21301

§ 371 Date: Sep. 2, 2022

Related U.S. Application Data
(60) Provisional application No. 63/055,050, filed on Jul. 22, 2020.
(60) Provisional application No. 62/985,995, filed on Mar. 6, 2020.

(51) Int. Cl. A61B 5/00; A61B 5/024; G16H 50/30

(52) U.S. Cl. CPC A61B 5/7275; A61B 5/024; G16H 50/30

(57) ABSTRACT

Provided are a system and method for predicting risk of diagnosis for Autism Spectrum Disorder (ASD) using neonatal analytics. Such analytics assess heart rate pattern data for a given period of admission in a Neonatal Intensive Care Unit to determine correlation with heart rate characteristics indicative of ASD. Relative to a finding of one or more correlations, such analytics offer the opportunity for earliest screening and intervention for ASD, as appropriate.

### CROSS-REFERENCE TO RELATED APPLICATIONS

This Application is a U.S. national stage filing under 35 U.S.C. § 371 of International Application No. PCT/US21/21301, filed Mar. 8, 2021, which claims priority to and the benefit of each of U.S. Provisional Application No. 62/985,995, filed Mar. 6, 2020, and U.S. Provisional Application No. 63/055,050, filed Jul. 22, 2020, the entire contents of each of such Applications being incorporated by reference herein.

### STATEMENT OF GOVERNMENT INTEREST

This invention was made with government support under Grant Nos. HL133708 and HD072071, awarded by The U.S. National Institutes of Health. The government has certain rights in the invention.

### FIELD OF THE DISCLOSURE

Disclosed embodiments relate to predicting risk of diagnosis, and more specifically, to predicting a risk of Autism Spectrum Disorder (ASD) diagnosis, during the course of an individual's lifespan that began with receipt of intensive medical care and through interpretation of analytics derived from such care.

### BACKGROUND

Citations throughout are to those documents referred to as References and listed at the conclusion of this section.

ASD, or autism, is generally recognized as a developmental disability affecting diagnosed individuals' abilities to communicate and interact with others in society, according to a variable degree of inability.⁴² Difficulties with thinking, learning, and problem-solving may be among those that beset such individuals.⁴²

The median age of diagnosis has been reported to be at about four (4) years of age,¹ and the U.S. Centers for Disease Control (CDC) reports an ASD identification rate of 1 in 54 for children eight (8) years of age, based on 2016 data.⁴³ ASD identification rose for similarly aged children when considering a reported rate of 1 in 59 from only two (2) years prior, i.e., 2014.⁴³ That is, the reported data reflects an increase in the incidence of ASD in the United States from about 1.7% to about 1.9%.

Aside from the observations above for the noted ages, it is known that infants who require and receive intensive care are inherently at increased risk for eventual diagnosis of ASD. Of these, risk of ASD diagnosis is quadrupled for preterm infants (i.e., gestational age (GA) of less than 37 weeks) relative to those of term.² A variety of conditions associated with preterm birth may contribute to this, including prenatal or neonatal inflammation.³ Fetal or neonatal hypoxia leading to white matter injury may also contribute to diagnosis.⁴ In term infants, hypoxic ischemic encephalopathy and other conditions requiring intensive care at birth are known to be associated with increased risk for later diagnosis of ASD.⁵⁻¹¹

Of measurable parameters, heart rate (HR), which is regulated by the autonomic nervous system, with sympathetic and parasympathetic activation leading to accelerations and decelerations, respectively, in response to internal or external stimuli,¹⁵ has exhibited differences in children, adolescents, and adults having ASD. In children, such differences have included variable HR patterns defining higher HR,¹⁶⁻¹⁷ overactive sympathetic tone,¹⁸⁻²⁰ and decreased parasympathetic or vagal tone, 21-25 when compared to neurotypical individuals. These pattern differences have been specifically noted during periods of sleep, and in response to social and non-social stimuli.²⁶

In these regards, societal benefit may be obtained for those most susceptible to risk for ASD diagnosis by taking advantage of study of HR pattern data as soon as it becomes available. As has been described above, the risk is prevalent among preterm infants, and term infants having at least the above-described conditions. Thus, for preterm infants and those of term requiring admission to a Neonatal Intensive Care Unit (NICU), such an environment, through its constant HR monitoring, offers the earliest possible opportunity for HR analysis.

As such, and in view of the benefits of inherently maximizing opportunity to provide expedited screening and intervention for ASD at an appropriate time subsequent to NICU discharge, it would be highly desirable to explore ways to examine neonatal HR pattern analytics to uncover possible links to a risk for ASD diagnosis. Doing so would allow such screening and intervention to occur at an earliest possible stage of brain development, and thus perhaps improve long-term outcomes for those eventually diagnosed.

### REFERENCES

1. Baio J, Wiggins L, Christensen D L, et al. Prevalence of Autism Spectrum Disorder Among Children Aged 8 Years—Autism and Developmental Disabilities Monitoring Network, 11 Sites, United States, 2014. MMWR Surveill Summ 2018; 67:1-23.
2. Agrawal S, Rao S C, Bulsara M K, Patole S K. Prevalence of Autism Spectrum Disorder in Preterm Infants: A Meta-analysis. Pediatrics 2018; 142.
3. Meldrum S J, Strunk T, Currie A, Prescott S L, Simmer K, Whitehouse A J O. Autism spectrum disorder in children born preterm-role of exposure to perinatal inflammation. Front Neurosci 2013; 7:123.
4. van Tilborg E, Achterberg E J M, van Kammen C M, et al. Combined fetal inflammation and postnatal hypoxia causes myelin deficits and autism-like behavior in a rat model of diffuse white matter injury. Glia 2018; 66:78-93.
5. Getahun D, Fassett M J, Peltier M R, et al. Association of Perinatal Risk Factors with Autism Spectrum Disorder. Am J Perinatol 2017; 34:295-304.
6. Lindquist B, Carlsson G, Persson E-K, Uvebrant P. Behavioural problems and autism in children with hydrocephalus: a population-based study. Eur Child Adolesc Psychiatry 2006; 15:214-9.
7. DiGuiseppi C, Hepburn S, Davis J M, et al. Screening for autism spectrum disorders in children with Down syndrome: population prevalence and screening test characteristics. J Dev Behav Pediatr 2010; 31:181-91.
8. Sigmon E R, Kelleman M, Susi A, Nylund C M, Oster M E. Congenital Heart Disease and Autism: A Case-Control Study. Pediatrics 2019; 144.
9. Xiang A H, Wang X, Martinez M P, et al. Association of maternal diabetes with autism in offspring. JAMA 2015; 313:1425-34.
10. Winkler-Schwartz A, Garfinkle J, Shevell M I. Autism spectrum disorder in a term birth neonatal intensive care unit population. Pediatr Neurol 2014; 51:776-80.
11. Marino B S, Lipkin P H, Newburger J W, et al. Neurodevelopmental outcomes in children with congenital heart disease: evaluation and management: a scientific statement from the American Heart Association. Circulation 2012; 126:1143-72.
12. Pelphrey K. Charting a course for autism biomarkers. Biol Psychiatry 2017; 82:155-6.
13. MacDonald R, Parry-Cruwys D, Dupere S, Ahearn W. Assessing progress and outcome of early intensive behavioral intervention for toddlers with autism. Res Dev Disabil 2014; 35:3632-44.
14. Rogers S J, Vismara L A. Evidence-based comprehensive treatments for early autism. J Clin Child Adolesc Psychol 2008; 37:8-38.
15. Axelrod F B, Chelimsky G G, Weese-Mayer D E. Pediatric autonomic disorders. Pediatrics 2006; 118:309-21.
16. Bujnakova I, Ondrejka I, Mestanik M, et al. Autism spectrum disorder is associated with autonomic underarousal. Physiol Res 2016; 65:S673-82.
17. Daluwatte C, Miles J H, Christ S E, Beversdorf D Q, Takahashi T N, Yao G. Atypical pupillary light reflex and heart rate variability in children with autism spectrum disorder. J Autism Dev Disord 2013; 43:1910-25.
18. Bharath R, Moodithaya S S, Bhat S U, Mirajkar A M, Shetty S B. Comparison of Physiological and Biochemical Autonomic Indices in Children with and without Autism Spectrum Disorders. Medicina (Kaunas) 2019; 55.
19. Billeci L, Tonacci A, Narzisi A, et al. Heart rate variability during a joint attention task in toddlers with autism spectrum disorders. Front Physiol 2018; 9:467.
20. Cheshire W P. Highlights in clinical autonomic neuroscience: new insights into autonomic dysfunction in autism. Auton Neurosci 2012; 171:4-7.
21. Sheinkopf S J, Levine T P, McCormick C E B, et al. Developmental trajectories of autonomic functioning in autism from birth to early childhood. Biol Psychol 2019; 142:13-8.
22. Ming X, Patel R, Kang V, Chokroverty S, Julu P O. Respiratory and autonomic dysfunction in children with autism spectrum disorders. Brain Dev 2016; 38:225-32.
23. Harder R, Malow B A, Goodpaster R L, et al. Heart rate variability during sleep in children with autism spectrum disorder. Clin Auton Res 2016; 26:423-32.
24. Schaaf R C, Benevides T W, Leiby B E, Sendecki J A. Autonomic dysregulation during sensory stimulation in children with autism spectrum disorder. J Autism Dev Disord 2015; 45:461-72.
25. Benevides T W, Lane S J. A review of cardiac autonomic measures: considerations for examination of physiological response in children with autism spectrum disorder. J Autism Dev Disord 2015; 45:560-75.
26. Perdue K L, Edwards L A, Tager-Flusberg H, Nelson C A. Differing developmental trajectories in heart rate responses to speech stimuli in infants at high and low risk for autism spectrum disorder. J Autism Dev Disord 2017; 47:2434-42.
27. Fairchild K D, Lake D E, Kattwinkel J, et al. Vital signs and their cross-correlation in sepsis and NEC: a study of 1,065 very-low-birth-weight infants in two NICUs. Pediatr Res 2017; 81:315-21.
28. Sullivan B A, Wallman-Stokes A, Isler J, et al. Early Pulse Oximetry Data Improves Prediction of Death and Adverse Outcomes in a Two-Center Cohort of Very Low Birth Weight Infants. Am J Perinatol 2018; 35:1331-8.
29. Vesoulis Z A, Bank R L, Lake D, et al. Early hypoxemia burden is strongly associated with severe intracranial hemorrhage in preterm infants. J Perinatol 2019; 39:48-53.
30. Harrell F E. Overview of maximum likelihood estimation. In: Regression modeling strategies: with applications to linear models, logistic and ordinal regression, and survival analysis. Cham: Springer International Publishing; 2015. p. 181-217.
31. White H. Maximum likelihood estimation of misspecified models. Econometrica 1982; 50:1.
32. Fairchild K, Mohr M, Paget-Brown A, et al. Clinical associations of immature breathing in preterm infants: part 1-central apnea. Pediatr Res 2016; 80:21-7.
33. Schneider U, Bode F, Schmidt A, et al. Developmental milestones of the autonomic nervous system revealed via longitudinal monitoring of fetal heart rate variability. PLoS One 2018; 13:e0200799.
34. Burtchen N, Myers M M, Lucchini M, Ordonez Retamar M, Rodriguez D, Fifer W P. Autonomic signatures of late preterm, early term, and full term neonates during early postnatal life. Early Hum Dev 2019; 137:104817.
35. Sahni R, Schulze K F, Kashyap S, Ohira-Kist K, Fifer W P, Myers M M. Maturational changes in heart rate and heart rate variability in low birth weight infants. Dev Psychobiol 2000; 37:73-81.
36. Porges S W, Furman S A. The early development of the autonomic nervous system provides a neural platform for social behaviour: a polyvagal perspective. Infant Child Dev 2011; 20:106-18.
37. Rother M, Witte H, Zwiener U, Eiselt M, Fischer P. Cardiac aliasing—a possible cause for the misinterpretation of cardiorespirographic data in neonates. Early Hum Dev 1989; 20:1-12.
38. Carvalho T D de, Massetti T, Silva T D da, et al. Heart rate variability in individuals with Down syndrome—A systematic review and meta-analysis. Auton Neurosci 2018; 213:23-33.
39. Dawson G, Bernier R. A quarter century of progress on the early detection and treatment of autism spectrum disorder. Dev Psychopathol 2013; 25:1455-72.
40. Douglas P S. Pre-emptive Intervention for Autism Spectrum Disorder: Theoretical Foundations and Clinical Translation. Front Integr Neurosci 2019; 13:66.
41. McPartland J C, Bernier R A, Jeste S S, et al. The Autism Biomarkers Consortium for Clinical Trials (ABC-CT): Scientific Context, Study Design, and Progress Toward Biomarker Qualification. Front Integr Neurosci 2020; 14:16.
42. https://www.cdc.gov/ncbddd/autism/facts.html.
43. Maenner M J, Shaw K A, Baio J, et al. Prevalence of Autism Spectrum Disorder Among Children Aged 8 Years—Autism and Developmental Disabilities Monitoring Network, 11 Sites, United States, 2016. MMWR Surveill Summ 2020; 69 (No. SS-4):1-12.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

Embodiments may include a system for predicting risk of diagnosis of Autism Spectrum Disorder (ASD) for an infant based on neonatal analytics sourced from one or more Neonatal Intensive Care Unit (NICU) records for said infant, including a processor, and a processor-readable memory including processor-executable instructions for receiving and storing heart rate (HR) pattern data corresponding to said infant of a predetermined postmenstrual age (PMA) for a predetermined time period; evaluating one or more parameters derived from said HR pattern data to assess a behavior of said one or more parameters within said predetermined time period; determining whether the behavior of any of said one or more parameters increases or decreases in magnitude relative to a datum; and in response to a determination of increasing behavior, determining that said risk is positive.

Additional embodiments may include a method and computer-readable medium relative to the aforementioned system.

In these regards, each of such embodiments may be further defined as provided below.

An aspect may provide that the predetermined time period includes one or more portions of time within a PMA of 34-42 weeks of the infant.

An aspect may provide that the one or more parameters include a measured HR standard deviation and a measured HR skewness (HRskw) each calculated for about ten (10) minute segments of HR pattern data and averaged on at least an hourly basis for each of the one or more portions of time within the PMA of 34-42 weeks of the infant.

An aspect may provide that the datum corresponds to any one of (a) a predetermined, respective HR or HRskw value and (b) a respective HR standard deviation or HRskw value for a preceding one of the one or more portions of time, within the PMA of 34-42 weeks, of equal duration.

An aspect may provide that an increase in the measured HRskw value is based on one or more accelerations in the HR pattern data.

In certain embodiments, the disclosed embodiments may include one or more of the features described herein.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIGS. 1(A)-1(C) illustrate representative HR Skewness (HRskw) for each of respective 10-minute HR tracings;

FIGS. 2(A)-2(B) illustrate HRskw relative to a variable number of weeks of postmenstrual age (PMA), and FIG. 2C illustrates HRskw at 34-42 PMA relative to risk of ASD diagnosis and observed occurrence thereof;

FIG. 3 illustrates a relative comparison of a percentage of HRskw values >1 with advancing PMA for each of males and females diagnosed with ASD and a control group not diagnosed with ASD;

FIG. 4 illustrates a schematic diagram of an apparatus operable to implement one or more aspects of embodiments herein; and

FIG. 5 illustrates a sequence diagram for predicting and assessing risk of ASD diagnosis according to embodiments herein.

### DETAILED DESCRIPTION

The present disclosure will now be described in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

It should also be noted that in some alternative implementations, the blocks in a flowchart, the communications in a sequence-diagram, the states in a state-diagram, etc., may occur out of the orders illustrated in the figures. That is, the illustrated orders of the blocks/communications/states are not intended to be limiting. Rather, the illustrated blocks/communications/states may be reordered into any suitable order, and some of the blocks/communications/states could occur simultaneously.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedure, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

It should be appreciated that any of the components or modules referred to with regards to any of the embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Ranges may be expressed herein as from “about” or “approximately” one particular value and/or to “about” or “approximately” another particular value. When such a range is expressed, other exemplary embodiments include from the one particular value and/or to the other particular value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

In an effort to predict risk of diagnosis for ASD, we, the present inventors group at the University of Virginia (UVa), sought to study analytics potentially signaling risk of eventual diagnosis of ASD so that earliest ASD screening and intervention may be sought. In doing so, our study differed from analyses pertinent to children, adolescents, and adults diagnosed with ASD by considering neonatal analytics. Such analytics were derived from a retrospective single-center cohort of Neonatal Intensive Care Unit (NICU) patients, with study focused on detection of increased HR patterning, which, as discussed above, has been identified in children diagnosed with ASD.

The cohort included, based on data ranging from 2009-2016, 2,371 NICU infants having available bedside monitor HR pattern data (HR data) and who had been seen in the UVa Health System beyond three (3) years of age, such that diagnosis for ASD in accordance with the median age of diagnosis of four (4) was captured. Of these, 88 infants representing four (4) percent of the cohort (74% of which were male), were confirmed to have been diagnosed with ASD, whereas the remainder of the cohort formed a control for which other developmental and behavioral disabilities were not excluded. Thus, the analytics derived from the HR data were verifiable.

The HR data was collected using the BEDMASTER system available from Excel Medical, and included electrocardiogram sampling every two (2) seconds (0.5 Hz). Values of zero were removed as incontrovertible artifact, and four (4) mathematical moments, representing HR data parameters and including mean, standard deviation, skewness, and kurtosis, were calculated in 10-minute segments and averaged each hour, thus representing predetermined time periods of evaluation of the HR data.

Table 1 below details various demographics for the cohort, wherein results are presented as median and interquartile range or number and percent unless otherwise noted. Demographic variables in infants with and without ASD were compared with the Wilcoxon rank-sum test for continuous variables and Fisher's exact test (two-sided) for categorical variables. Maternal diabetes included Types I and II and gestational diabetes. Congenital cardiac malformation included those diagnosed on echocardiogram during the NICU stay, excluding atrial septal defect and patent ductus arteriosus. Diagnosis of brain injury included severe intraventricular hemorrhage (Grade III or IV), cystic periventricular leukomalacia, hypoxic-ischemic encephalopathy, cerebral infarct or thrombosis, and EEG-confirmed seizures. Diagnosis of brain malformation included congenital hydrocephalus and other malformations diagnosed on brain MRI during the NICU stay. Among the demographics are the p-value corresponding to the area under the Receiver Operator Characteristics (ROC) curve, or AUC, and indicating a greater degree of statistical significance according to a given, lower p-value.

TABLE 1

| | Total | | Autism | | Control | | |
|---|---|---|---|---|---|---|---|
| | n = 2371 | | n = 88 | | n = 2283 | | p = |
| Male | 1333 | (56%) | 65 | (74%) | 1268 | (56%) | 0.001 |
| GA (weeks) | 35 | (31,38) | 35 | (33, 38) | 35 | (31, 38) | 0.750 |
| Premature (<37 weeks GA) | 1444 | (61%) | 55 | (63%) | 1389 | (61%) | 0.824 |
| Extremely premature (<28 weeks GA) | 266 | (11%) | 11 | (13%) | 255 | (11%) | 0.730 |
| Birth Weight (kg) | 2.4 | (1.6, 3.2) | 2.4 | (1.6, 3.2) | 2.4 | (1.6, 3.2) | 0.713 |
| VLBW (<1500 g) | 520 | (22%) | 19 | (22%) | 501 | (22%) | 1.000 |
| Small for GA (<10th % ile) | 319 | (13%) | 15 | (17%) | 304 | (13%) | 0.338 |
| Twin or Triplet | 387 | (16%) | 21 | (24%) | 366 | (16%) | 0.056 |
| Maternal age (years) | 27 | (23, 32) | 28 | (22, 33) | 27 | (23, 32) | 0.942 |
| Maternal race white | 1770 | (75%) | 65 | (76%) | 1705 | (79%) | 0.589 |
| Maternal race black | 441 | (19%) | 19 | (22%) | 422 | (20%) | 0.489 |
| Maternal ethnicity Hispanic | 168 | (7%) | 6 | (7%) | 162 | (7%) | 1.000 |
| Maternal diabetes | 40 | (2%) | 1 | (1%) | 39 | (2%) | 1.000 |
| C-section delivery | 1238 | (52%) | 43 | (51%) | 1195 | (54%) | 0.508 |
| Apgar 5 minute | 8 | (7, 9) | 8 | (7, 9) | 8 | (7, 9) | 0.450 |
| Trisomy 21 | 41 | (2%) | 9 | (10%) | 32 | (1%) | <0.001 |
| Congenital cardiac malformation | 278 | (12%) | 9 | (10%) | 269 | (12%) | 1.000 |
| Brain injury or malformation | 232 | (10%) | 8 | (9%) | 224 | (10%) | 1.000 |
| Day of age at discharge home | 15 | (6, 35) | 16 | (9, 37) | 15 | (6, 35) | 0.838 |

median (25th, 75th % ile) or n (%)

Upon inspection of the above, ASD was statistically significantly associated with male sex and Trisomy 21 (both p≤0.001).

With reference to Table 2 below, a multivariate logistic regression model correcting for baseline variables including birthweight (BW), gestational age (GA), sex, and Trisomy 21 predicted future diagnosis of ASD with area under the ROC curve, or AUC, of 0.637. In this multivariate model, BW and GA were not statistically significant. Trisomy 21 and male sex had odds ratios (95% Confidence Interval (CI)) of 8.05 and 2.21 for ASD, respectively.

Analysis of the four (4) mathematical moments derived from the cohort data is shown in Table 2 below for a postmenstrual age (PMA) of 34-42 weeks (discussed below), and based on a median of 182 hours of HR data per infant (i.e., interquartile range (IQR) of 63,376), with like coverage for ASD and control infants. As demonstrated, mean and kurtosis were not significantly different for the two groups; yet, standard deviation (SD) and skewness (skw) were, with skewness being most strongly indicative of ASD. These observations maintained when HR was assessed individually (HR Metric), and became strengthened when adding the multivariate logistically regressed baseline model correcting for sex, GA, BW, Trisomy 21, and PMA.

TABLE 2

| | | | | | Hr Metric | | HR + Baseline Model | |
|---|---|---|---|---|---|---|---|---|
| Mathematical | Control (n = 2283) | | Autism (n = 88) | | ROC | | ROC | |
| Moment | mean | SD | mean | SD | AUC | p = | AUC | p = |
| HR Mean | 153 | 15 | 153 | 16 | 0.491 | 0.972 | 0.666 | 0.678 |
| HR SD | 8.16 | 3.31 | 9.11 | 4.01 | 0.569 | 0.014 | 0.676 | 0.009 |
| HR Skewness | −0.07 | 0.74 | 0.10 | 0.70 | 0.566 | 0.004 | 0.684 | 0.002 |
| HR Kurtosis | 5.08 | 3.18 | 4.80 | 3.21 | 0.540 | 0.211 | 0.670 | 0.238 |

Baseline Model includes sex, GA, BW, Trisomy 21, and PMA

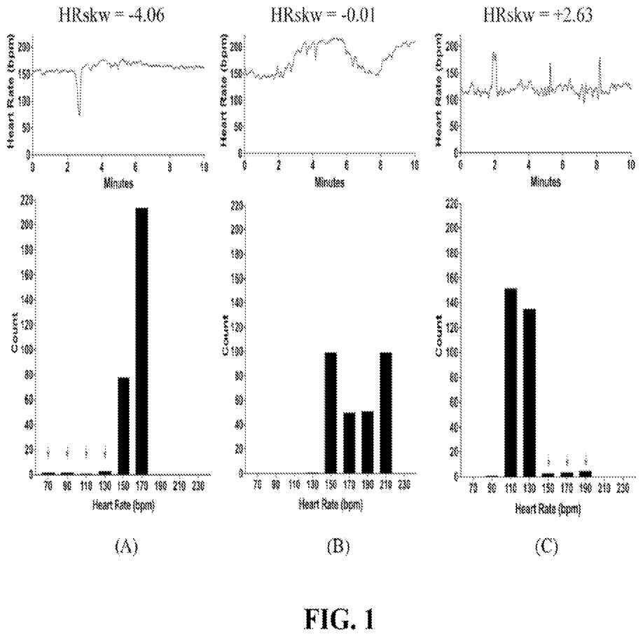

As is understood, skewness is a representation of asymmetry of a histogram. With reference to FIG. 1, representations of such asymmetry may be seen whereas, with respect to shown 10-min HR tracings therein, (a) low HRskw is indicative of HR decelerations (see FIG. 1A), (b) high HRskw is indicative of HR accelerations (see FIG. 1C), and (c) owing to the fact that skewness is sensitive to outlier values (indicated by arrows in FIGS. 1A and 1C), gradual increases or decreases in HR yield minimal skewness effect (see FIG. 1B).

Because preterm infants (GA<37 weeks) have been identified as having HR decelerations partly due to apnea of prematurity, such decelerations may be reflected as negative skewness. Thus, it is appropriate to examine a period when such condition begins to significantly diminish so as to identify indicators of ASD including sympathetic overactivity and parasympathetic underactivity likely represented by HR accelerations, i.e., increased HRskw. We have identified that period as including a PMA of 34-42 weeks (see Table 2 above).

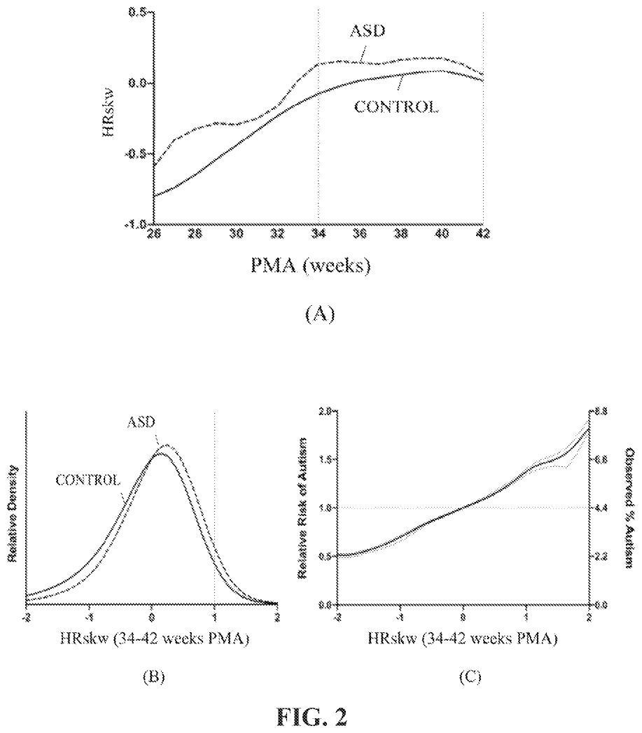

In support of the findings tabulated in Table 2, reference may be made to FIGS. 2A to 2C relating the relative risk of ASD to HRskw as regards PMA. In particular, FIG. 2A demonstrates that HRskw behaves so as to increase with advancing PMA and only begins to level off after 34 weeks PMA, as to both ASD and the control group. FIG. 2B demonstrates a density plot of all hourly HRskw values during 34-42 weeks PMA which relatively increase in number for ASD when contrasted with the control group. Finally, FIG. 2C shows positive linear relationships between HRskw and relative risk of diagnosis of ASD, in view of observed percentage (risk=1 as to 4.4% occurrence).

When assessing for differential as to sex, the data showed that males are at significantly higher risk of being diagnosed with ASD than are females, relative to a highest percentile of HRskw measurements. In referring to Table 3 below, which demonstrates HRskw relative to rate and risk of ASD diagnosis based on HRskw percentiles from 34-42 weeks PMA (as assessed from four (4) days of NICU HR data), rate of diagnosis in the highest 5^{th} percentile was 11.1% for males, as contrasted with 3.7% for females. Overall rates of diagnosis for males and females were 5.1% and 2.5%, respectively. Statistical significance was adjusted for repeated measures using the Huber-White method for robust covariance estimation.³⁰⁻³¹ Statistical analyses were performed in GRAPHPAD PRISM and MATLAB with two-tailed p<0.05 being considered statistically significant.

TABLE 3

| HR | HR | | | | | | | | | | | | |
|---|---|---|---|---|---|---|---|---|---|---|---|---|---|
| skewness | skewness | All (0.570, 0.06) | | | | Male (0.600, 0.02) | | | | Female (0.524, 0.73) | | | |
| percentile | range | n | Autism | Rate | Rel. Risk | n | Autism | Rate | Rel. Risk | n | Autism | Rate | Rel. Risk |
| <25^{th} % ile | −1.83 to −0.16 | 405 | 9 | 2.22% | 0.56 | 229 | 5 | 2.18% | 0.43 | 176 | 4 | 2.27% | 0.92 |
| 25^{th} to 95^{th} % ile | −0.16 to 0.55 | 1132 | 48 | 4.24% | 1.07 | 648 | 36 | 5.56% | 1.10 | 484 | 12 | 2.48% | 1.00 |
| >95^{th} % ile | 0.55 to 2.22 | 81 | 7 | 8.64% | 2.18 | 54 | 6 | 11.11% | 2.20 | 27 | 1 | 3.70% | 1.50 |
| ALL | −1.83 to 2.22 | 1618 | 64 | 3.96% | 1 | 931 | 47 | 5.05% | 1 | 687 | 17 | 2.47% | 1 |

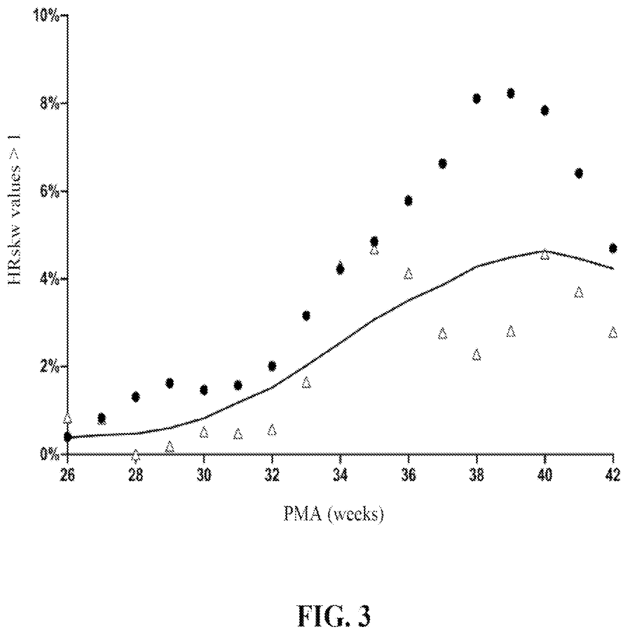

AUC,p-value shown in parentheses ( ) as to All, Male, and Female cohort members having assessed NICU HR data for at least four (4) days; thus, cohort size equals 1618, with 47 males and 17 females diagnosed with ASD. Data showed average HRskw as being significant at 0.008 when correcting for baseline risk factors, and demonstrated an AUC of 0.664 in contrast to 0.637 as to only such factors. The disparity is illustrated in FIG. 3 comparing percentage of HRskw values greater than (>) 1 relative to PMA in weeks, wherein males are represented by depicted darkened circles (•) and females by unfilled triangles (Δ).

With particular reference to at least FIG. 3, it may be seen that a greater occurrence of HRskw indicating HR accelerations occurred in the male, but not female, cohort population for the period of 34-42 weeks PMA.

As noted above, skewness toward more HR accelerations comports with developmental maturation, given that apnea of prematurity declines after about 34 weeks PMA, and may be representative of imbalance in autonomic activation often associated with ASD in children. Such observation was undisturbed as the control group included infants later diagnosed with other significant neurodevelopmental conditions such as global development delay, attention deficit hyperactivity disorder, and Trisomy 21, which is commonly associated with autonmic instability and elevated risk for ASD diagnosis. That is, the finding that HRskw toward more HR accelerations during the period of 34-42 weeks PMA substantially supports the conclusion that elevated HRskw is not a non-specific risk indicator of neurodevelopmental disorder, and is thus likely associable with risk for eventual diagnosis of ASD. This is buttressed by the observation of Table 3 and FIG. 2C that relative risk of ASD diagnosis increases as a magnitude for HRskw increases. That said, it may thus be observed that such risk decreases relative to decreased HRskw. In these regards, such increase or decrease may be measured against a given datum, including, for example, one or more preceding portions of equal duration of a PMA period. Alternatively, such datum may comprise a predetermined HRskw value. Such datum measures may equally apply with respect to standard deviation of HR pattern data insofar as standard deviation may be measured temporally as in the case of HRskw or against a predetermined value therefor.

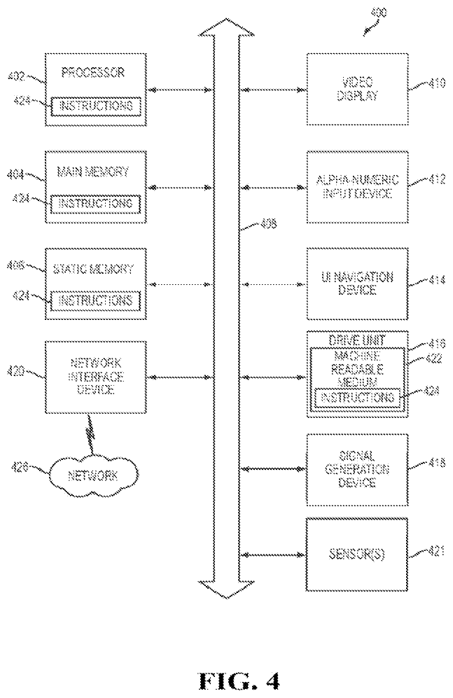

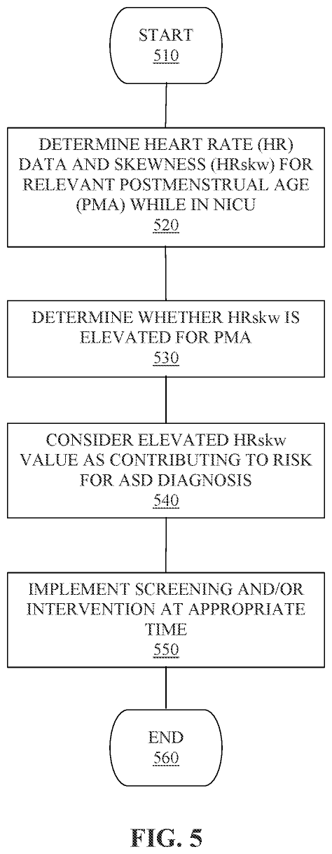

In these regards, and with reference to FIG. 4, there is illustrated an apparatus for implementing a method of predicting risk of diagnosis for ASD using neonatal analytics as discussed with regard to FIG. 5 below.

Therein, the apparatus may include a machine 400 that may include logic, one or more components, and circuits (e.g., modules). Circuits may be tangible entities configured to perform certain operations. In an example, such circuits may be arranged (e.g., internally or with respect to external entities such as other circuits) in a specified manner. In an example, one or more computer systems (e.g., a standalone, client or server computer system) or one or more hardware processors (processors) may be configured with or by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, may cause the circuit to perform the certain operations.

In an example, a circuit may be implemented mechanically or electronically. For example, a circuit may comprise dedicated circuitry or logic that may be specifically configured to perform one or more techniques such as are discussed above, including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” may be understood to encompass a tangible entity, whether physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiple of such circuits exist contemporaneously, communications may be achieved through signal transmission (e.g., over appropriate circuits and buses) that connect the circuits. In embodiments in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a collection of information.

The various operations of methods described herein may be performed, at least partially, by one or more processors that may temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor-implemented. For example, at least some of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) may be implemented in digital electronic circuitry, in computer hardware, in firmware, in software, or in any combination thereof. Example embodiments may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus can be implemented as, special purpose logic circuitry (e.g., a field programmable gate array (FPGA) or an application-specific integrated circuit (ASIC)).

The computing system or systems herein may include clients and servers. A client and server may generally be remote from each other and generally interact through a communication network. The relationship of client and server arises by virtue of computer programs running on the respective computers and having a client-server relationship to each other. In embodiments deploying a programmable computing system, it will be appreciated that both hardware and software architectures may be adapted, as appropriate. Specifically, it will be appreciated that whether to implement certain functionality in permanently configured hardware (e.g., an ASIC), in temporarily configured hardware (e.g., a combination of software and a programmable processor), or a combination of permanently and temporarily configured hardware may be a function of efficiency. Below are set out hardware (e.g., machine 400) and software architectures that may be implemented in or as example embodiments.

In an example, the machine 400 may operate as a standalone device or the machine 400 may be connected (e.g., networked) to other machines.

In a networked deployment, the machine 400 may operate in the capacity of either a server or a client machine in server-client network environments. In an example, machine 400 may act as a peer machine in peer-to-peer (or other distributed) network environments. The machine 400 may be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the embodiments discussed herein.

Example machine (e.g., computer system) 400 may include a processor 402 (e.g., a central processing unit (CPU), a graphics processing unit (GPU) or both), a main memory 404 and a static memory 406, some or all of which may communicate with each other via a bus 408. The machine 400 may further include a display unit 410, an alphanumeric input device 412 (e.g., a keyboard), and a user interface (UI) navigation device 411 (e.g., a mouse). In an example, the display unit 410, input device 412 and UI navigation device 414 may be a touch screen display. The machine 400 may additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 may include a machine readable medium 422 on which is stored one or more sets of data structures or instructions 424 (e.g., software) embodying or utilized by any one or more of the methodologies or functions described herein. The instructions 424 may also reside, completely or at least partially, within the main memory 404, within static memory 406, or within the processor 402 during execution thereof by the machine 400. In an example, one or any combination of the processor 402, the main memory 404, the static memory 406, or the storage device 416 may constitute machine readable media.

While the machine readable medium 422 is illustrated as a single medium, the term “machine readable medium” may include a single medium or multiple media (e.g., a centralized or distributed database, and/or associated caches and servers) that may be configured to store the one or more instructions 424. The term “machine readable medium” may also be taken to include any tangible medium that may be capable of storing, encoding, or carrying instructions for execution by the machine and that cause the machine to perform any one or more of the embodiments of the present disclosure or that may be capable of storing, encoding or carrying data structures utilized by or associated with such instructions. The term “machine readable medium” may accordingly be understood to include, but not be limited to, solid-state memories, and optical and magnetic media. Specific examples of machine readable media may include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 may further be transmitted or received over a communications network 426 using a transmission medium via the network interface device 420 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” may include any intangible medium that may be capable of storing, encoding or carrying instructions for execution by the machine, and includes digital or analog communications signals or other intangible medium to facilitate communication of such software.

In referring to FIG. 5, there is shown a relational sequence of steps for implementation of the method of predicting a risk of diagnosis of ASD when using neonatal analytics, according to embodiments discussed herein. Thus, and whereas the sequence may initiate at step 510, a determination, as informed from bedside monitoring in the NICU and sourced from one or more periodic records of NICU admission, including an entire length of such admission, may be made at step 520 as to HR pattern data and an associated, measured HRskw for the relevant PMA, and namely 34-42 weeks PMA. Proceeding to step 530, a determination may then be made as to whether HRskw value(s) for a given infant are elevated for the given PMA. In this regard, elevation may be assessed according to mathematical techniques as are discussed herein with respect to a distribution of HR data collected for a given infant during that infant's NICU admission. Once a determination has been made, the same may be weighed relative to, for instance, the findings herein, such that, at step 540, consideration may be given, in conjunction with other conditions and information pertinent to the individual infant, that a derived, elevated HRskw value may be a contributing indication of risk of ASD diagnosis. Thereafter, at step 550, appropriate timing for screening and/or intervention for ASD may be contemplated accordingly, whereafter the sequence, as set forth herein, concludes at step 560.

Thus, as may be appreciated from the above by one of skill in the art, we, the present UVa inventors group, have set forth manner of predicting risk of diagnosis for ASD based on neonatal analytics when focusing on HR pattern data and skewness derived therefrom. In these regards, we have identified that increased HRskw indicative of increased HR accelerations is particularly supportive of increased risk for eventual diagnosis of ASD, especially among the male NICU population. Accordingly, we have also, therefore, signaled that the NICU population may benefit from the analyses described herein so that earliest ASD screening and intervention may occur, including adaptations therefor, as appropriate.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

What is claimed is:

1. A system for predicting risk of diagnosis of Autism Spectrum Disorder (ASD) for an infant based on neonatal analytics sourced from one or more Neonatal Intensive Care Unit (NICU) records for said infant, comprising:
a processor;
a processor-readable memory including processor-executable instructions for:
receiving and storing heart rate (HR) pattern data corresponding to said infant of a predetermined postmenstrual age (PMA) for a predetermined time period,
evaluating one or more parameters derived from said HR pattern data to assess a behavior of said one or more parameters within said predetermined time period,
determining whether the behavior of any of said one or more parameters increases or decreases in magnitude relative to a datum, and
in response to a determination of increasing behavior, determining that said risk is positive.

2. The system according to claim 1, wherein:
said predetermined time period comprises one or more portions of time within a PMA of 34-42 weeks of said infant.

3. The system according to claim 2, wherein:
said one or more parameters comprise a measured HR standard deviation and a measured HR skewness (HRskw) each calculated for about ten (10) minute segments of HR pattern data and averaged on at least an hourly basis for each of said one or more portions of time within said PMA of 34-42 weeks of said infant.

4. The system according to claim 3, wherein:
said datum corresponds to any one of (a) a predetermined, respective HR standard deviation or HRskw value and (b) a respective HR standard deviation or HRskw value for a preceding one of said one or more portions of time, within said PMA of 34-42 weeks, of equal duration.

5. The system according to claim 4, wherein:
an increase in the measured HRskw value is based on one or more accelerations in said HR pattern data.

6. A processor-implemented method for predicting risk of diagnosis of Autism Spectrum Disorder (ASD) for an infant based on neonatal analytics sourced from one or more Neonatal Intensive Care Unit (NICU) records for said infant, comprising:
receiving and storing in a memory heart rate (HR) pattern data corresponding to said infant of a predetermined postmenstrual age (PMA) for a predetermined time period,
evaluating one or more parameters derived from said HR pattern data to assess a behavior of said one or more parameters within said predetermined time period,
determining whether the behavior of any of said one or more parameters increases or decreases in magnitude relative to a datum, and
in response to a determination of increasing behavior, determining that said risk is positive.

7. The method according to claim 6, wherein:
said predetermined time period comprises one or more portions of time within a PMA of 34-42 weeks of said infant.

8. The method according to claim 7, wherein:
said one or more parameters comprise a measured HR standard deviation and a measured HR skewness (HRskw) each calculated for ten (10) minute segments of HR pattern data and averaged on at least an hourly basis for each of said one or more portions of time within said PMA of 34-42 weeks of said infant.

9. The method according to claim 8, wherein:
said datum corresponds to any one of (a) a predetermined, respective HR standard deviation or HRskw value and (b) a respective HR standard deviation or HRskw value for a preceding one of said one or more portions of time, within said PMA of 34-42 weeks, of equal duration.

10. The method according to claim 9, wherein:
an increase in the measured HRskw value is based on one or more accelerations in said HR pattern data.

11. A non-transient computer-readable medium having stored thereon computer-readable instructions for predicting risk of diagnosis of Autism Spectrum Disorder (ASD) for an infant based on neonatal analytics sourced from one or more Neonatal Intensive Care Unit (NICU) records for said infant, said instructions comprising instructions causing a computer to:
receive and store in a memory heart rate (HR) pattern data corresponding to said infant of a predetermined postmenstrual age (PMA) for a predetermined time period,
evaluate one or more parameters derived from said HR pattern data to assess a behavior of said one or more parameters within said predetermined time period,
determine whether the behavior of any of said one or more parameters increases or decreases in magnitude relative to a datum, and
in response to a determination of increasing behavior, determine that said risk is positive.

12. The computer-readable medium according to claim 11, wherein:
said predetermined time period comprises one or more portions of time within a PMA of 34-42 weeks of said infant.

13. The computer-readable medium according to claim 12, wherein:
said one or more parameters comprise a measured HR standard deviation and a measured HR skewness (HRskw) each calculated for ten (10) minute segments of HR pattern data and averaged on at least an hourly basis for each of said one or more portions of time within said PMA of 34-42 weeks of said infant.

14. The computer-readable medium according to claim 13, wherein:
said datum corresponds to any one of (a) a predetermined, respective HR standard deviation or HRskw value and (b) a respective HR standard deviation or HRskw value for a preceding one of said one or more portions of time, within said PMA of 34-42 weeks, of equal duration.

15. The computer-readable medium according to claim 14, wherein:
an increase in the measured HRskw value is based on one or more accelerations in said HR pattern data.
